(19) United States
(12) Patent Application Publication
(10) Pub. No.: US 2016/0101899 A1
(43) Pub. Date: Apr. 14, 2016

(54) INVENTORY SUPPLY SYSTEM AND METHODS OF USE

(71) Applicant: Christian D. Weidner, Minnetonka, MN (US)

(72) Inventor: Christian D. Weidner, Minnetonka, MN (US)

(21) Appl. No.: 14/877,591

(22) Filed: Oct. 7, 2015

Related U.S. Application Data
(60) Provisional application No. 62/061,510, filed on Oct. 8, 2014.

(51) Int. Cl. B65D 25/04; G06F 19/00; G06Q 10/08

(52) U.S. Cl. CPC B65D 25/04; G06Q 10/087; G06F 19/327

(57) ABSTRACT

An inventory supply system and method of use to effectively manage equipment and supply inventory. Specifically there is a need for a precise control and data acquisition system that can be easily implemented and readily used for a large number of medical devices and supplies contained in a single area.

### CROSS REFERENCE TO RELATED APPLICATIONS

This application claims the benefit of U.S. Provisional Application No. 62/061,510, filed Oct. 8, 2014, entitled “Inventory Supply System and Methods of Use,” which is incorporated by reference herein in its entirety.

### BACKGROUND

There is a need to track supplies and the current inventory within healthcare institutions. These include sterile and non-sterile medical devices and supplies. The medical devices and supplies include diapers, personal care items, syringes, needles, diapers, and the like. In some institutions, medical devices are contained within secured areas to allow access to authorized personnel. These secured regions allow for medical devices to be stored in an area, whether it be an entire room or a small cabinet, that has precise physical parameter control(s), including control of temperature, pressure, humidity, and the like. Many regulatory agencies also require reporting of adverse events, such as when the temperature exceeds an acceptable level. Thus, the limited access to the medical devices and supplies provides protection against contamination.

Medical device and supply vendors spend countless hours sending staff to hospitals and clinics to replenish supplies that are needed. Conversely, hospitals and clinics may employ facility personnel that are tasked with the duty to constantly manage inventory. When a particular device or supply is needed, the facility personnel can order the supplies. These supplies are then delivered. However, the use of external vendors or facility personnel to manage supply rooms is cumbersome and inefficient. Further, the need for non-healthcare providers to enter supply rooms poses a risk of contamination of the medical devices and supplies.

Examples of healthcare facilities include hospitals, nursing homes, home-health care organizations, a research facility, a health care clinic, a doctor's office, government facility, a surgery center and a medical center.

Other industries also employ similar inventory management protocols, which are equally inefficient. Industries such as automotive, chemical, transportation, food supply, pharmaceutical, aerospace, mechanical and electrical are a non-exclusive list of industries that employ such protocols.

Thus there is a need in the art for systems, methods and apparatus for effectively managing equipment inventory. Specifically there is a need for a precise control and data acquisition system that can be easily implemented and readily used for a large number of medical devices and supplies contained in a single area.

### BRIEF DESCRIPTION OF THE DRAWINGS

FIG. 1 is a top perspective view of an embodiment of the Inventory Supply System.

FIG. 2 is a front elevation view of the Inventory Supply System illustrated in FIG. 1.

FIG. 3 is a rear elevation view of the Inventory Supply System thereof.

FIG. 4 is a right side elevation view of the Inventory Supply System thereof.

FIG. 5 is a left side elevation view of the Inventory Supply System thereof.

FIG. 6 is a top plan view of the Inventory Supply System thereof.

FIG. 7 is a bottom plan view of the Inventory Supply System thereof.

FIG. 8 is a top perspective view of the Inventory Supply System in a down configuration.

FIG. 9 is a top perspective view of the Inventory Supply System in a container shown in an upright configuration.

FIG. 10 is a top perspective view of the Inventory Supply System in a container shown in a down configuration.

FIG. 11 is a top perspective view of the Inventory Supply System in a container shown in an upright configuration.

FIG. 12 is a top perspective view of the Inventory Supply System in a container shown in a down configuration.

FIG. 13 is a top perspective view of multiple Inventory Supply Systems with data center system.

### DESCRIPTION

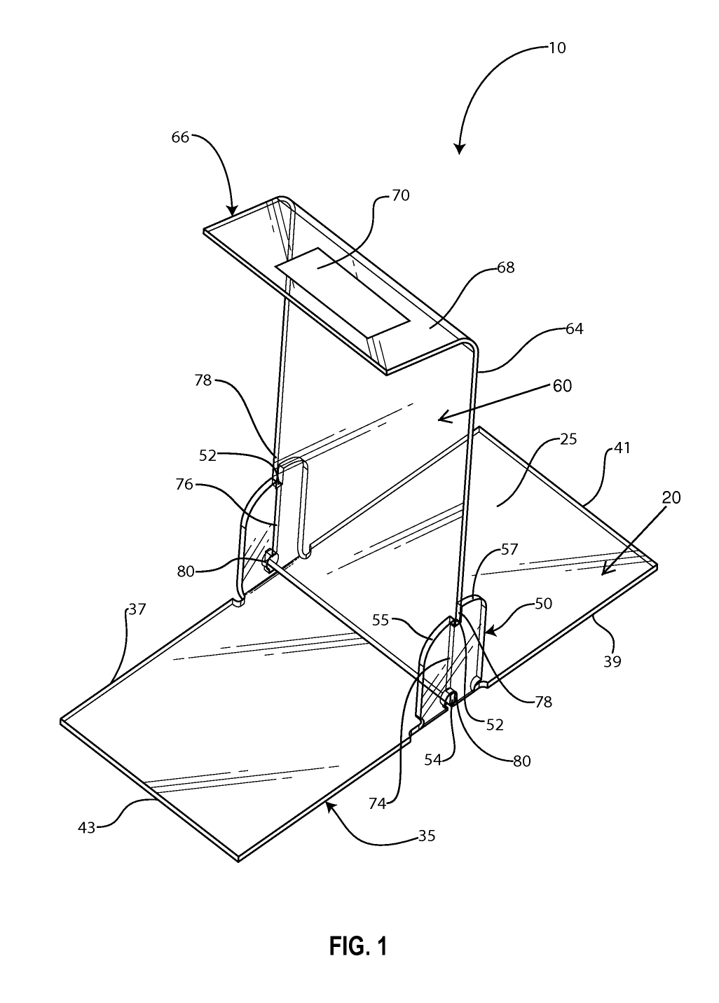

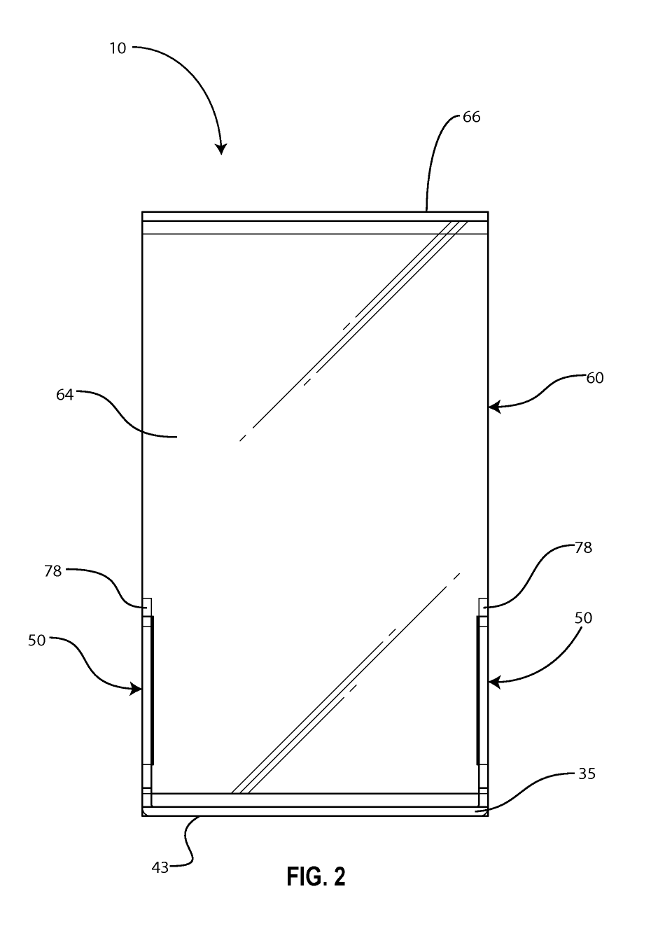

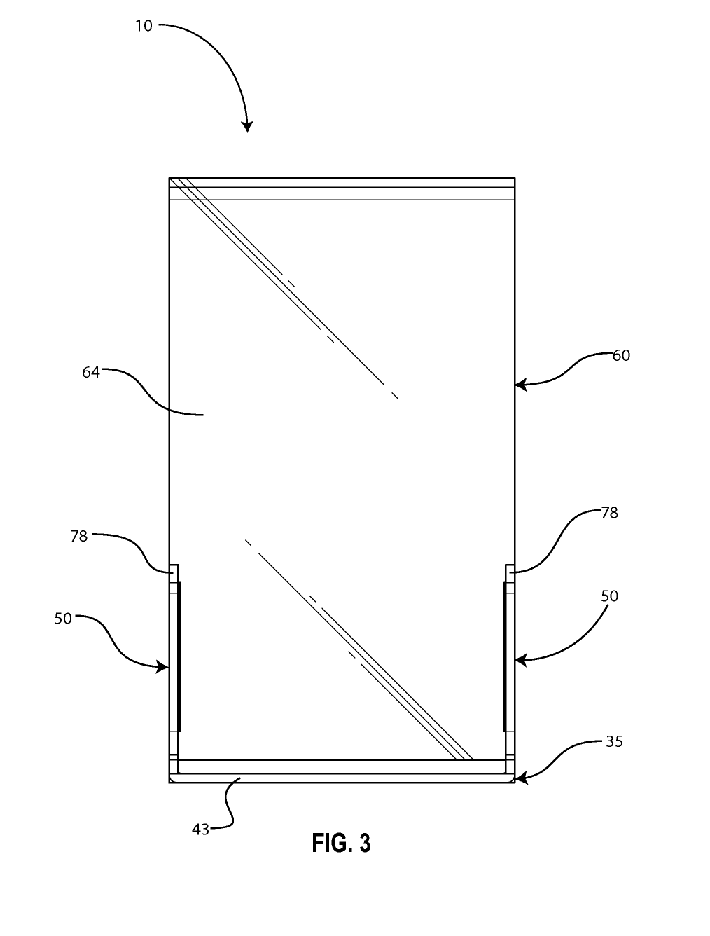

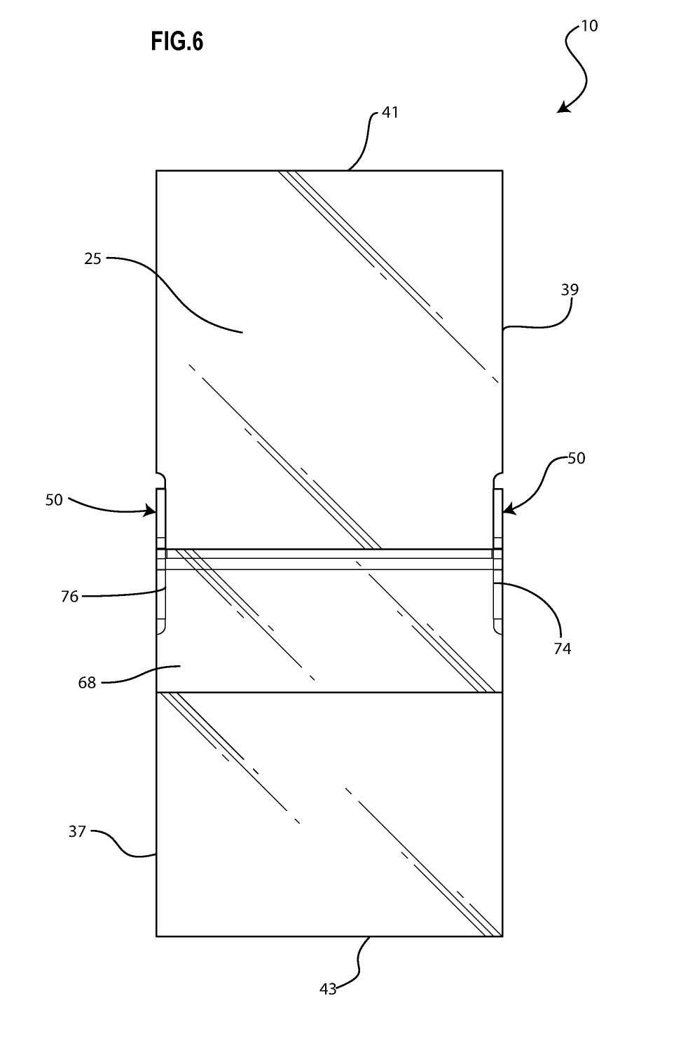

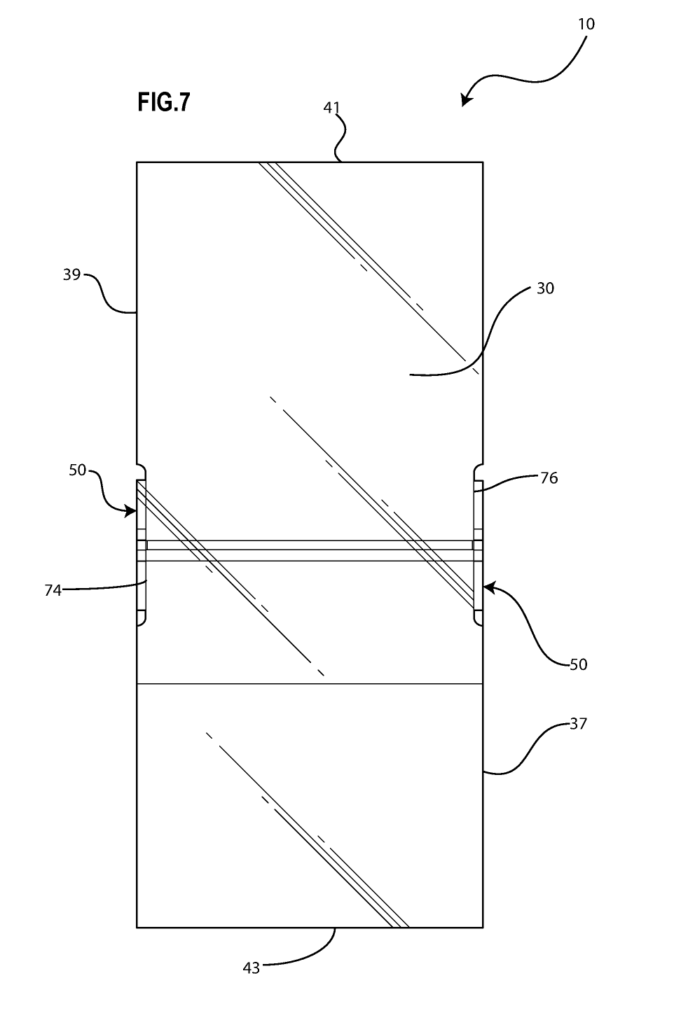

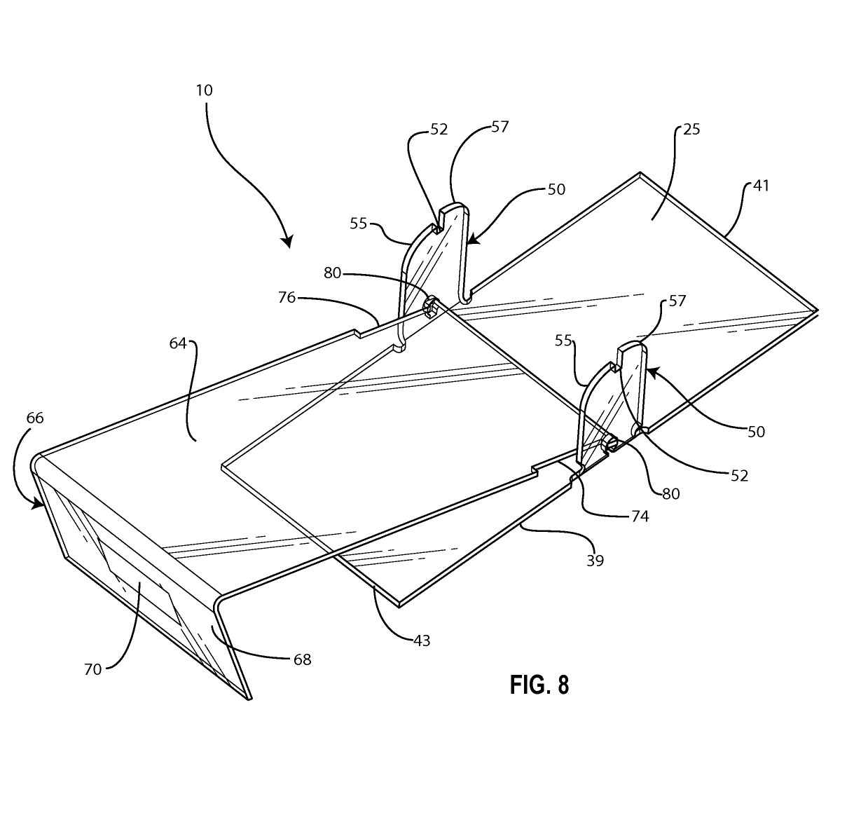

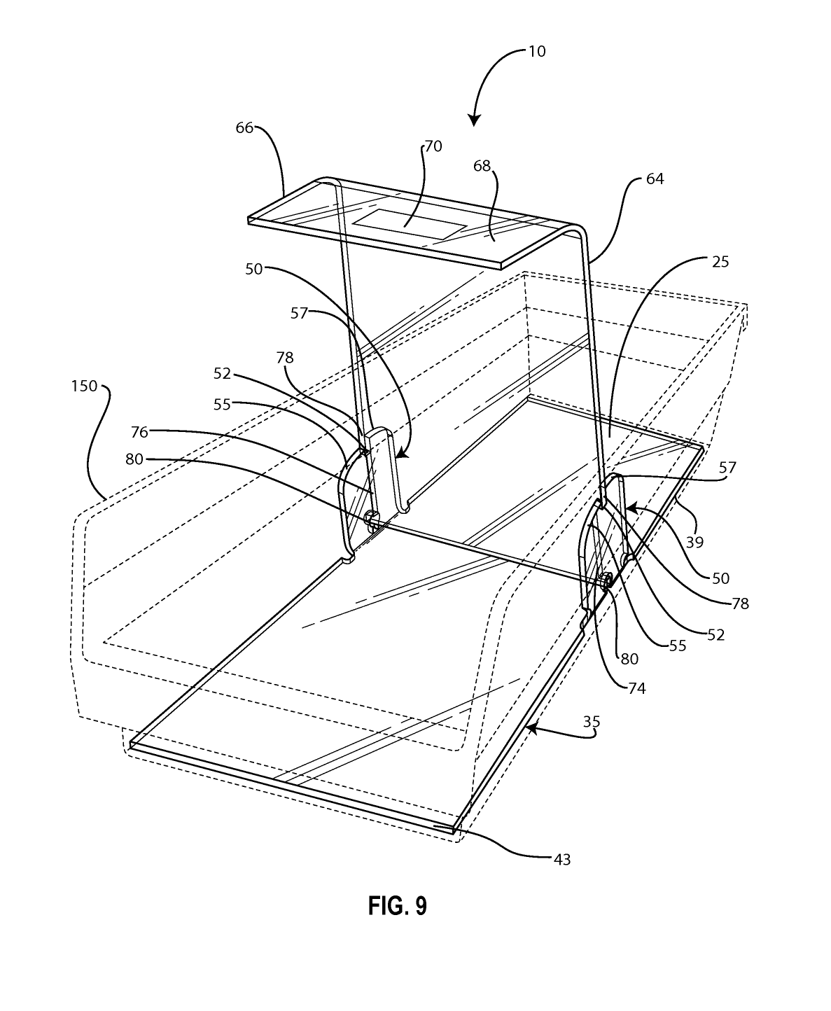

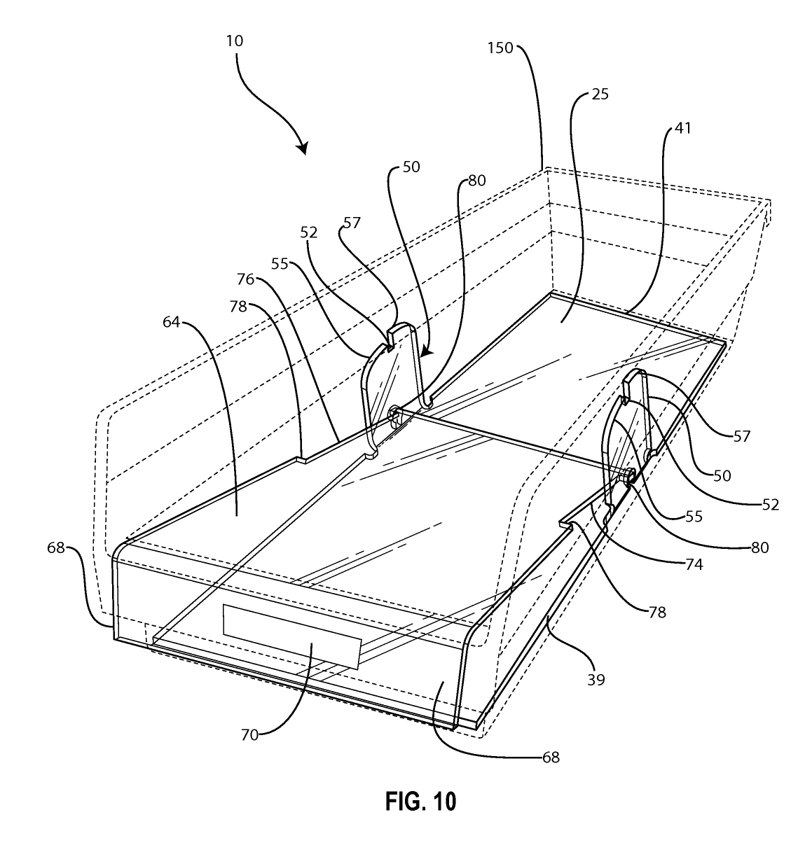

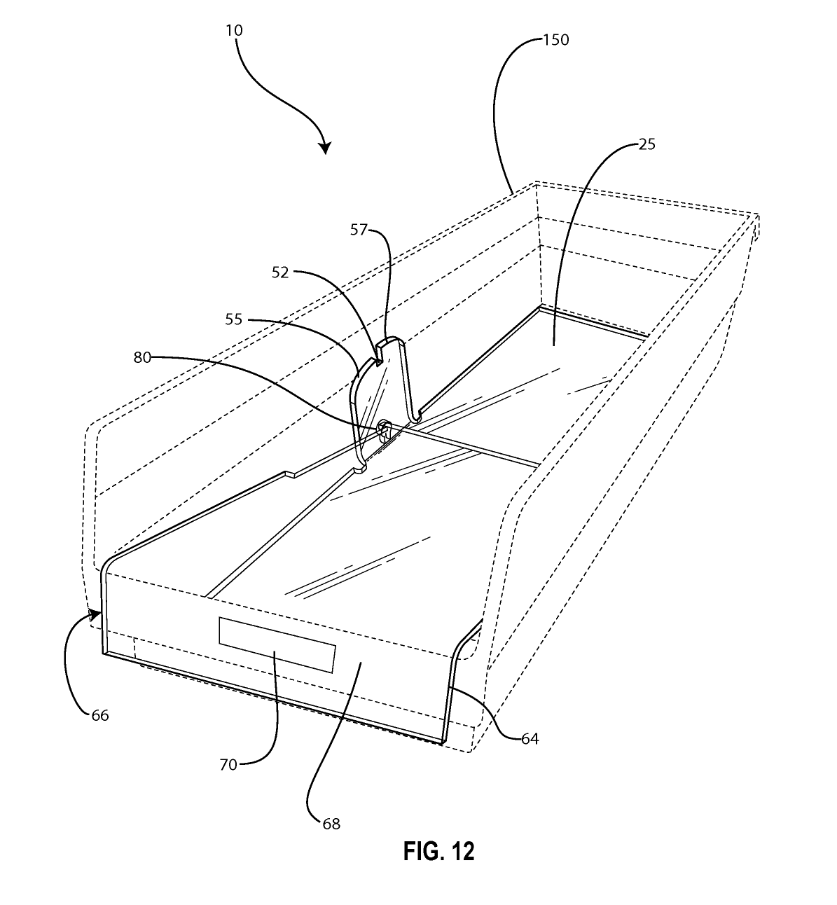

Referring now to the drawings, wherein like reference numerals designate identical or corresponding parts throughout the several views, FIGS. 1-12 illustrate one embodiment of an inventory supply system 10. As shown in FIG. 1, inventory supply system 10 generally includes a base 20, a locking plate 50 and a divider 60. Inventory supply system 10 may be made of a material comprising silicon, polyurethane, rubbers, neoprene, nylon, PVC, polystyrene, polyethylene, polypropylene and the like. Other materials, such as nanomaterials and composite plastics are also within the scope of the invention. The material may optionally include an antimicrobial material. In some example embodiments, the material is autoclavable. Although not wanting to be bound by any particular theory, inventory supply system 10 is made of a material that can be sterilized and is lightweight.

Referring now to FIGS. 1 and 6-7 which illustrates base 20 having top side 25, a bottom side 30 connected by a terminal edge 35 comprising; a left edge 37, right edge 39, front edge 43 and back edge 41. Base 20 is fabricated in a manner to allow for a substantially level surface to dispose inventory supply system 10 along the bottom of a container, described more fully below. As shown, base 20 is a substantially solid planar structure; however, other modifications to base 20 are within the scope of the invention. For example, in an alternative embodiment, base 20 may include apertures throughout to allow for access to the bottom side 30 to clean or sterilize base 20. In at least this example embodiment, the base 20 may have a screen or mesh appearance. It should be appreciated that base 20 may be designed in several ways so long as base 20 can provide a stable surface for which to dispose inventory supply system 10 when in use.

Divider 60, as best shown in FIGS. 1-5 and 8, generally includes a main body portion 64 and a blocking surface 68 which, as shown, is integrally related to main body portion 64 and forms a continuous surface between the two related parts. Main body portion 64 is planar having two recesses 74, 76 shown on opposing sides of main body portion 64. Recesses 74, 76 allow for main body portion 64 to engage a locking plate 50.

Front portion 66 comprises a blocking surface 68 that contains a signal block 70 which is used to cover signaling means 72 (i.e. a receiver or transmitter) when in use and as explained in further detail below. Front portion 66 must be fashioned in a manner to allow for coverage of signaling means 72 to prevent a signal from reaching the data center when in inventory supply system 10 is in a lowered position.

Signal block 70 is positioned on the blocking surface 68 of divider 60. Signal block 70 may be integrally related to blocking surface 68 such that the two respective pieces form a single unitary structure. As shown, signal block 70 is disposed upon the outer surface of front portion 66. Signal block 70 may be made of a material such as carbon steel, aluminum, composites, ceramics, gels, liquids and the like. The material should be able to block the signaling means 72 from either receiving or sending communication to data center.

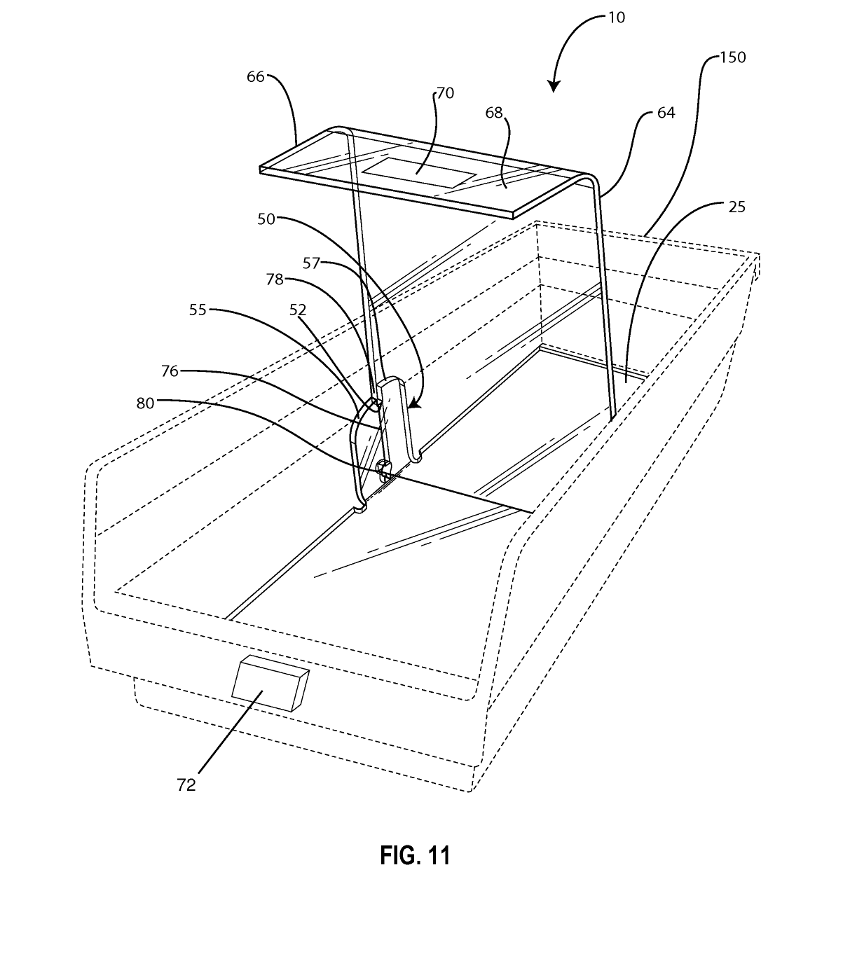

Referring now to FIG. 11 where signaling means 72 is positioned along the front wall of supply container 150 and is capable of generating a signal, such as light, radio waves, magnetic, electromagnetic, etc. Signaling means 72 may be permanently fixed to supply container 150 or removably coupled. In embodiments where signaling means 72 is permanently fixed to supply container 150, there is a need to ensure that the proper supplies are contained within the particular supply container associated with a particular signaling means 72. Conversely, in embodiments where signaling means 72 is removably disposed on supply container 150, the signaling means 72 must be attached to a container with the correct supplies contained therein. Thus, a user can establish inventory protocols to provide data center and signaling means the ability to cross-check that the proper supplies are contained within a specific supply container. To state another way, both example embodiments have metrics to give the user the ability to have a particular supply correspond with a particular signaling mean 72 which is essential for data center 200 to initiate notification and/or order initiation sequence which corresponds to the correct supply.

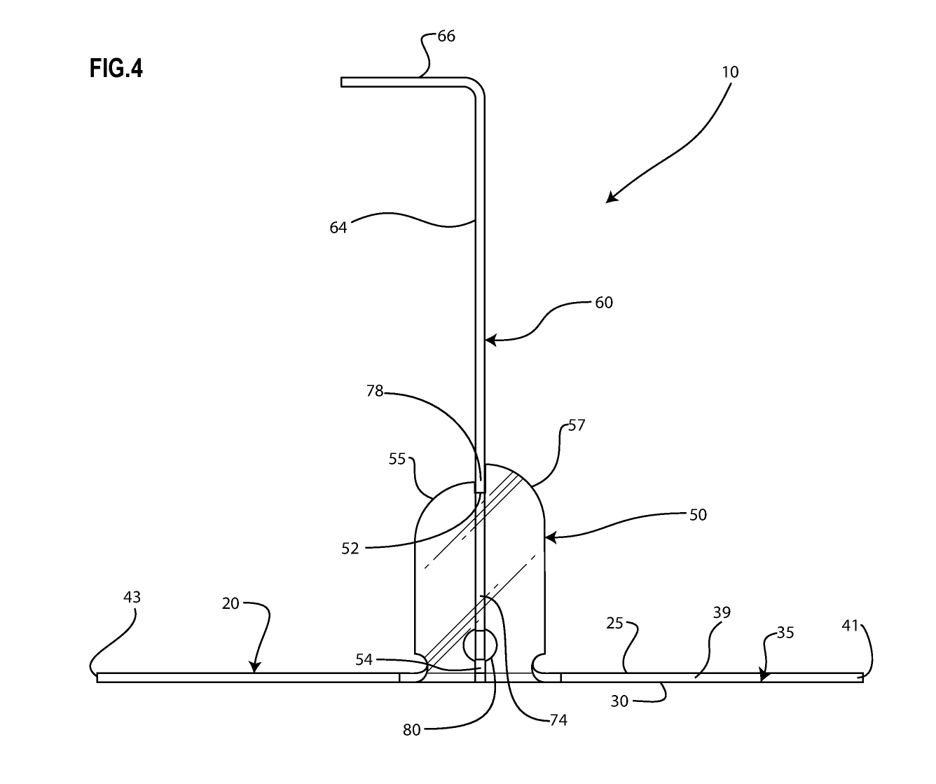

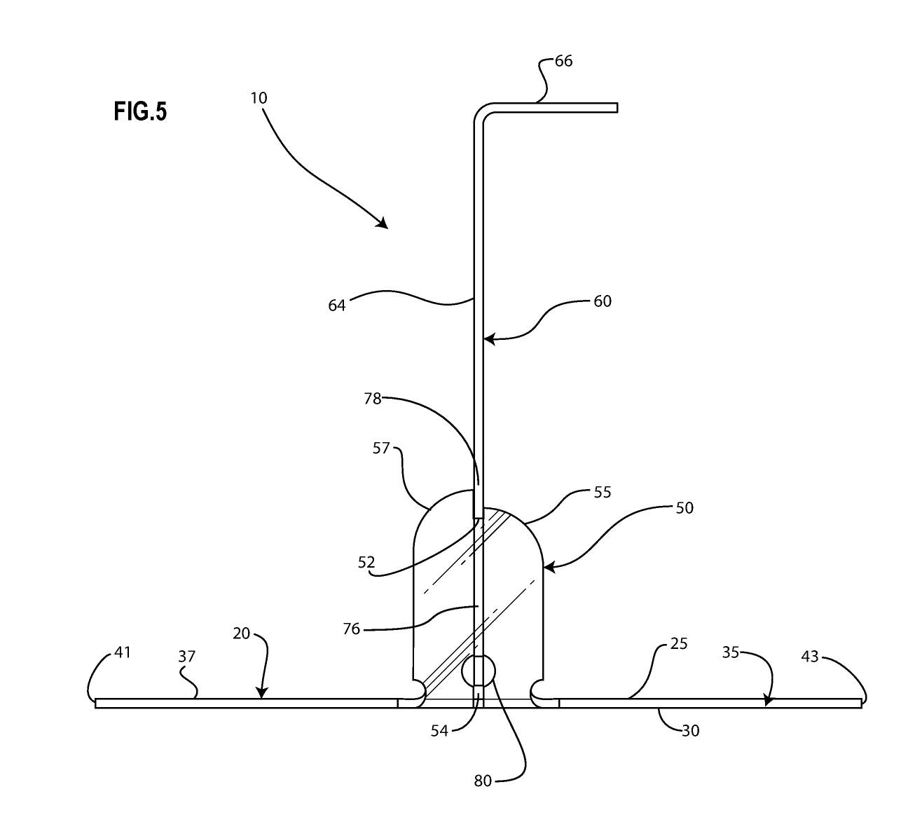

Referring again to FIGS. 1, 4-5 and 8 wherein locking plate 50 is perpendicularly configured in relation to base 20. Locking plate 50 is shown having a front arch 55, a back arch 57, an upper locking groove 52 and a lower locking groove 54. Although not necessary, locking plate 50 may be formed from a single unitary structure with base 20. As best shown in FIGS. 4 and 5, back arch 57 is higher than front arch 55 to provide support to divider 60 when in an upright configuration. Further, front arch 55 being shorter will provide less travel before the top notch 78 and bottom notch 80 are removed from upper locking groove 52 and lower locking groove 54 respectively. It should be appreciated that the height differential between front arch 55 and back arch 57 allows for divider 60 to move in a forward position, but not backwards from the substantially vertical position.

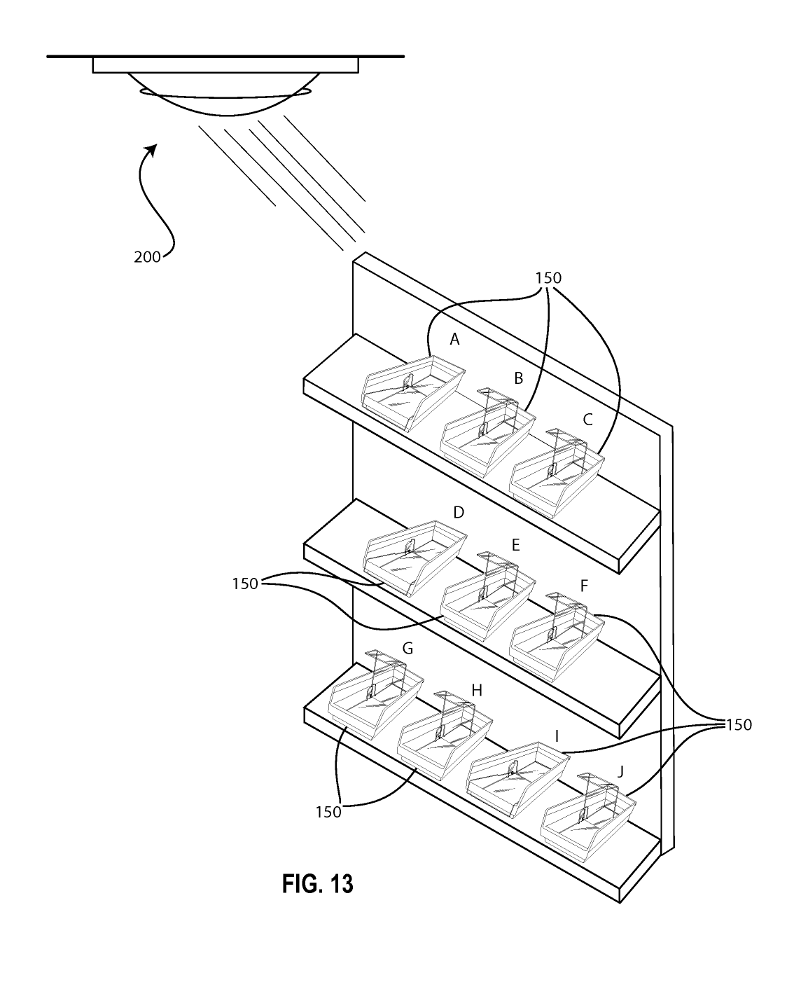

Referring now to FIG. 13 which illustrates a data center 200. Data center 200 includes a number of systems known by those of ordinary skill in the art, and may include software, hardware, wireless communication means, and the like. When in use, inventory supply system 10 will have a signaling means 72 that is unique for a particular supply container 150 and/or supply. Signaling means may include wireless and wired communication, a non-exclusive list of communication means include: RFID, radio, ultrasonic, infrared, free-space optical communication, light, magnetic, electromagnetic and electronic magnetic induction. Data center 200 and the accompanying systems are capable of calibrating data obtained from signaling means 72. In accordance with at least one example embodiment, there is provided a method that includes establishing set points for acceptable differences between baseline control data and comparison data, for differences between consecutive baseline data to accommodate the end user with metrics for managing supply inventory. For example, data center 200 may open a means for receiving a communication from signaling means 72 at a predetermined time interval. This time interval could be constant, or turn on and off in select time intervals, such as seconds, minutes, hours or days between the activation of receiving means. The time interval will depend on the need and frequency of supply usage. When data center 200 is available to receive a signal, data signals will be collected from the inventory supply room from each of the activated signaling means 72.

As illustrated in FIG. 13 data center 200 activates a receiving means which will detect signals from bins B, C, E, F, G, H and J. In this scenario, data center 200 will document that the supplies in supply containers A, D, and I are now at approximately 50%. This will in turn initiate a notification prompt to the user to order or refill the supplies contained within A, D and I. It should be appreciated that the program utilized for inventory management may include a modified receiving time interval (i.e. 5 minutes) when data center 200 does not receive a signal from a particular signaling means to verify that the divider 60 has not been inadvertently knocked down by someone desiring a particular supply. This feature will prevent a false prompt in the situation where the divider 60 was accidently placed down, but ultimately placed back in an upright position within a relatively short period of time. This additional verification step will help to reduce the number of false activation prompts to the user.

In an alternative embodiment, data center 200 may open a means for receiving communication from signaling means 72 at a predetermined time interval. When data center 200 is available to receive a signal data, signals will be collected from the inventory supply room from each of the activated signaling means 72. In the illustration in FIG. 13 data center 200 activates a receiving means which will detect signals from bins A, D, and I, but not B, C, E, F, G, H and J. In this scenario, data center 200 will document that the supplies in supply containers A, D, and I are now at approximately 50%. This will in turn initiate a notification prompt to the user. However, in this example, the moving of divider 60 to a down position will move a portion of supply container 150 and the signaling means 72 will be able to send a communication to data center 200. Similar to above, the program may optionally utilize a modified receiving time interval (i.e. 5 minutes) to verify that the divider 60 has not been inadvertently knocked down by someone desiring a particular supply, but who ultimately places the divider 60 back in an upright position, which would in turn recover the signaling mean.

Although a number of initiation prompts may be utilized, when data center receives a prompt that a particular supply container 150 is in need of additional supplies, data center 200 may facilitate system supply ordering and may be programmed to directly order a particular good from a vendor. Moreover, the data center 200 may collect data as to the amount of additional supplies that may be located in another storage room and prompt facility personnel to resupply a particular supply container 150 in a particular room. Additionally, the data center 200 may also track the shipping order to provide metrics for the user to access the timeliness of delivery or status of supplies in transit.

The example methods provided herein describe an inventory supply system 10 that is capable of generating additional metrics to provide a supply room with an environment conducive to maintaining supply life and usability. For example, in at least one example embodiment, data center 200 may optionally include sensing probe(s) (not shown) to determine the temperature and other related physical data (e.g., humidity, pressure, etc.) on a periodic basis for monitoring and reporting purposes of an supply room or a particular supply container 150 (via signaling means data).

Data center 200 inventory analysis of the supply container 150 may monitor inventory, monitor the healthcare provider who accessed the supply container when the divider was moved down, and/or monitor the facility employee who stocked the supply container 150. Thus, the data center 200 will be in communication with a central server or the like (either directly via a LAN line or wirelessly) that may be accessible by multiple sources including device manufacturers, regulatory agencies, hospital administration, and the like.

While particular examples of the inventory supply system are illustrated and described herein, those skilled in the art will understand that other configurations of the inventory supply system may be implemented. The foregoing description is presented to enable one of ordinary skill in the art to make and use the invention and is provided in the context of a patent application and its requirements. Various modifications to the preferred embodiment of the apparatus, and the general principles and features of the system and methods described herein will be readily apparent to those of skill in the art. Thus, the present invention is not to be limited to the embodiments of the apparatus and methods described herein and illustrated in the drawing figures, but are to be accorded the widest scope consistent with the spirit and scope of the appended claims.

1. A inventory supply system comprising;
a base being substantially planar;
a divider connected to the base having a main body portion and a front portion wherein the front portion further comprises a signal block; and
a locking plate for releasably engaging the divider.

2. The inventory supply system of claim 1 further comprising a signaling means.

3. The inventory supply system of claim 1, wherein the signal block comprises a material selected from a group consisting of ferrous metals, non-ferrous metals, metal alloys, aluminum, beryllium, bismuth, brass, cadmium, carbon steel, ceramic, cerium, cobalt, composites, copper, gels, gold, iron, lead, liquids, mercury, nickel, platinum, polytetrafluoroethylene, silver, stainless steel, steel, tin, titanium, tungsten and zinc.

4. The inventory supply system of claim 1, wherein the inventory supply system further comprises a supply container for placing a portion of the base therein.

5. The inventory supply system of claim 4, wherein the inventory supply system further comprises a signaling means connected to the supply container.

6. The inventory supply system of claim 5, wherein the signaling means is selected from a group consisting of RFID, radio, ultrasonic, infrared, free-space optical communication, light, magnetic, electromagnetic and electronic magnetic induction.

7. The inventory supply system of claim 2, wherein the signaling means is selected from a group consisting of RFID, radio, ultrasonic, infrared, free-space optical communication, light, magnetic, electromagnetic and electronic magnetic induction.

8. The inventory supply system of claim 2, wherein the signaling means is capable of generating a signal selected from a group consisting of RFID, radio, ultrasonic, infrared, free-space optical communication, light, magnetic, electromagnetic and electronic magnetic induction.

9. The inventory supply system of claim 1, wherein the locking plate comprises at least one locking groove for positioning a portion of the divider therein.

10. The inventory supply system of claim 1, wherein the locking plate comprises an upper locking groove and a lower locking groove for positioning a portion of the divider therein.

11. A inventory supply system comprising;
a base being substantially planar;
a divider connected to the base having a main body portion and a front portion wherein the front portion further comprises a signal block;
a locking plate for releasably engaging the divider;
a supply container; and
a signaling means.

12. The inventory supply system of claim 11, wherein said signal block comprises a material selected from a group consisting of ferrous metals, non-ferrous metals, metal alloys, aluminum, beryllium, bismuth, brass, cadmium, carbon steel, ceramic, cerium, cobalt, composites, copper, gels, gold, iron, lead, liquids, mercury, nickel, platinum, polytetrafluoroethylene, silver, stainless steel, steel, tin, titanium, tungsten and zinc.

13. A method for using an inventory supply system the step comprising;
positioning an inventory supply system within a supply container, wherein the inventory supply system comprises a base being substantially planar; a divider connect to the base having a main body portion and a front portion wherein the front portion further comprises a signal block;
and a locking plate for releasably engaging the divider; and
filling the supply container with supplies wherein the divider being in a substantially vertical has supplies within the container in front and behind the divider.

14. The method of claim 13, wherein the supply container further comprises a signaling means connected thereto.

15. The method of claim 14, wherein the inventory supply system further comprises a data center.

16. The method as claimed in claim 15, wherein the data center is in communication with the signaling means.

17. The method of claim 16 further comprising the step of moving the divider from the substantially vertical position to a forward position.

18. The method of claim 17, wherein moving the divider from the substantially vertical position to the forward position thereby blocks the communication between the data center and the signaling means.

19. The method of claim 15, wherein the data center further comprises at least one environmental probe for determining at least one physical data element selected from the group consisting of temperature, pressure and humidity.

20. The method of claim 19, wherein the at least one environmental probe is a temperature probe.

21. The method of claim 18 further comprising the step of initiating a signal to fill the supply container with supplies.
